(19) United States
(12) Patent Application Publication
(10) Pub. No.: US 2012/0028253 A1
(43) Pub. Date: Feb. 2, 2012

(54) METHOD FOR AMPLIFYING OLIGONUCLEOTIDE AND SMALL RNA BY USING POLYMERASE-ENDONUCLEASE CHAIN REACTION

(75) Inventors: Xiaolong Wang, Shandong (CN); Cuixian I.V., Shandong (CN); Deming Gou, Shandong (CN); Chenguang Liu, Shandong (CN)

(73) Assignee: Xiaolong WANG

(21) Appl. No.: 13/133,384

(22) PCT Filed: Apr. 3, 2009
(86) PCT No.: PCT/CN2009/000362

§ 371 Date: Sep. 2, 2011

(30) Foreign Application Priority Data
Jan. 5, 2009 (CN) 200910300070.8

(51) Int. Cl. C12Q 1/68; C12P 19/34

(52) U.S. Cl. 435/6.11; 435/91.2; 435/91.21

(57) ABSTRACT

A method for amplifying oligonucleotide in vitro by polymerase-endonuclease chain reaction (PECR) which utilizes a single-stranded DNA probe containing repeat sequences, extends a target oligonucleotide by a thermostable DNA polymerase, cleaves extended products with a thermostable endonuclease, and amplifies target oligonucleotide by thermocycling. In PECR, a specific oligonucleotide is exponentially amplified using one single probe instead of a pair of primers, and the reaction is precisely controlled by thermal cycles whose parameters are flexibly adjustable according to length, sequence, melting temperature and initial amount of the target oligonucleotide. Amplification speed depends totally on initial amount of target oligonucleotide present in the reaction system. The method can be used to amplify specific small nucleic acids, such as oligonucleotides and microRNAs, and further conduct quantitative analysis. PECR is easy to conduct with high efficiency, specificity and stability, and thus can be widely used in molecular biology studies.

### BACKGROUND OF THE PRESENT INVENTION

1. Field of Invention

The present invention relates to the field of molecular biology and gene engineering. More specifically, the present invention is a method for amplifying oligonucleotides and small RNA molecules.

2. Description of Related Arts

Nucleic acid amplification technology is at the core of contemporary molecular biology and gene engineering. In recent years, with the emerging of novel nucleic acid amplification methods, many new nucleic acid amplification based detection and diagnosis approaches have been developed and widely used. Although these methods have encountered some problems in practice, such as false positives and false negatives, they have many advantages, especially for small amount of sample requirement, rapid, sensitive and accurate. Therefore, many researchers from worldwide dedicated in developing new nucleic acid amplification methods, or improving existing technologies.

According to whether the reaction temperature changes or not, nucleic acid amplification methods can be divided into two broad categories: respectively thermocycling amplification methods and isothermal amplification methods. Classical thermocycling amplification methods include polymerase chain reaction (PCR) and ligase chain reaction (LCR); recent emerging isothermal amplification methods mainly include strand displacement amplification (SDA), rolling circle amplification (RCA), loop-mediated amplification (LAMP), helicase-dependent isothermal amplification (HDA), nucleic acid sequence based amplification (NASBA), and transcription-based amplification system (TAS), and so on.

Making a comprehensive survey at current nucleic acid amplification technologies in molecular biology, molecular diagnosis and gene engineering, in spite of many novel nucleic acid amplification approaches has been reported, PCR (U.S. Pat. Nos. 4,683,195 and 4,683,202) is still the most commonly used method for in vitro nucleic acid amplification. PCR and reverse transcription PCR (RT-PCR) is simple and effective for amplification of DNA and mRNA with sufficient length. But it is not able to be used directly for amplifying small nucleic acids, such as oligonucleotides, microRNAs (miRNAs) and small interfering RNAs (siRNAs). The reverse transcript of a miRNA is actually an oligonucleotide that is generally only 18-25 nucleotides in length. Therefore, the length of miRNA-derived cDNA is usually insufficient for designing a pair of specific primers, which is a prerequisite of PCR.

Oligonucleotides have a wide range of applications in modern molecular biology studies. Although a large quantity of a synthetic oligonucleotide that have an identical sequence can be accurately quantified by spectrophotometry analysis according to its optical density (OD), amplification and quantification of trace amount of target oligonucleotide in a biological sample that contains a lot of different nucleic acid sequences, is still an unsolved technical problem, which may have important applications in science. For example, it can be used for preparation of oligonucleotides and quantitative analysis of small RNAs such as miRNA and siRNA.

However, only a few studies on the amplification of oligonucleotides have been reported. The international patent application, PCT/US04/02718, entitled “Isothermal Reactions for the Amplification of Oligonucleotides”, described the Exponential Amplification Reaction (EXPAR) that is capable of amplifying oligonucleotides. The method relies on polymerase, nicking enzymes and strand displacement, using a repeat-containing single-stranded DNA (ssDNA) as template, 10⁷-fold amplification of the target oligonucleotide can be achieved in 5 min. The reaction is very easy, fast, and carried out at constant temperature conditions, without the need of a PCR instrument. However, in a recent study it is pointed out that serious non-specific background amplification and false positives exist in the reaction (Tan E, et al, Specific versus nonspecific isothermal DNA amplification through thermophilic polymerase and nicking enzyme activities. Biochemistry 2008, 47 (38): 9987-9999). Hence, the method is unreliable for use in quantitative analysis of nucleic acids.

On the other hand, microRNA, or miRNA in short, is a class of endogenous small regulatory RNA, about 20-24 nucleotides in length. In 1999, miRNA lin-4 was firstly found in nematodes. Since then, a lot of miRNAs with important roles in gene regulation were found in worms, fruit flies, mice, zebra fish and other model organisms and humans. MiRNA binds to its target, protein coding messenger RNA (mRNA), in the 3′-untranslated region (3′-UTR) with full complementarility inducing target mRNA degradation, or with partial complementarility inhibiting translation of the target mRNA, which is called post-transcriptional gene silencing (PTGS). MiRNAs involve in the regulation processes, and play important roles in the basic life activities in a lot of organisms. For example, lin-4 involved in controlling nematodes timing of larval development, mir-14 controls Drosophila cell death and fat metabolism. In Zebrafish, miR-214 determines the fate of muscle cells, and miR-430 functions the removal of maternal mRNAs that are no longer needed in early embryos. MiR-375 is a highly conserved islet-specific miRNA family. In Zebrafish, miR-375 determines islet development. Reduced levels of miR-375 inhibited the aggregation of islet cells and insulin secretion in humans. The role of miR-375 in other model organisms and humans is highly conserved, suggesting that the function of miR-375 is conservative from Zebrafish to human. The functional importance of miRNAs has attracted researchers worldwide to study the origin, mechanism and function of miRNA using a variety of model organisms.

As of Mar. 6, 2009, the miRNA database (miRbase Release 12.0) has a collection of 8619 miRNA sequences. However, in contrast to the rapid discovery of new miRNAs, progress in miRNA functional studies is far slower. By far there are only a few miRNAs whose function is well characterized. For functional miRNA research, first is to determine the target genes of the miRNA and its regulatory function, second is to study the temporal and spatial expression of its own regulation by quantitative detection. Since the timing and the tissue-specificity of miRNA expression is useful in revealing their function in a specific tissue and cell. The main cause of the slow progress in miRNA functional studies has two points: firstly, it is difficult to determine their target genes; secondly, the amplification and quantification of small RNAs is far more difficult than that of long messenger RNAs.

At present, amplification and detection of miRNA are based on real time quantitative reverse transcription-PCR (qRT-PCR). Because mature miRNAs have no poly (A) tails, reverse transcription-PCR methods for miRNA are different from that for mRNA. There are mainly two strategies: first, miRNAs is polyadenalated using Poly (A) Polymerase (PAP) to add poly (A) tails to the 3′-end, and then synthesis cDNA by using Oligo (dT) and reverse transcriptase. Another method is using a miRNA-specific primer (miSP) for reverse transcription. The miSP contains a specific sequence complementary to the 3′-end of the miRNA and a stem loop structure. Since miRNA is too short to design a pair of specific primers, therefore, no matter which method is used, reverse transcription must be conducted using a downstream primer containing a universal tag, by which the universal tag is introduced into the cDNA. And then the resulting cDNA is amplified by PCR using a miRNA-specific primer as upstream primer and a universal primer as downstream primer.

The efficiency of polyadenalation and reverse transcription is of crucial importance to the accuracy of quantitative analysis. However, using an extra-long primer with a universal tag for reverse transcription will cause a decline in the efficiency of reverse transcription, and thus have a negative impact on the accuracy of the quantification of the miRNA. In addition, in PCR, the difference of the melting temperatures (Tm) between the upstream and the downstream primers should not exceed more than 2° C. However, for many miRNA-specific primers, their Tm inevitable differ more than 5° C. from the universal primer, because a same universal primer is not fit for all miRNA sequences. Some miRNA-specific primers and the universal primer will bind to each other to form primer dimers, resulting in non-specific amplification, false positive and false negative problems.

### SUMMARY OF THE PRESENT INVENTION

Current available nucleic acid amplification methods, such as PCR, have difficulties in amplification of oligonucleotides and small RNAs. This invention presents a new nucleic acid amplification method, designated as polymerase-endonuclease chain reaction (PECR), or polymerase-endonuclease amplification reaction (PEAR). PECR uses only one single ssDNA probe to amplify a specific miRNA or a target oligonucleotide. The PECR method comprises utilizing a repeat-containing ssDNA probe, repeatedly extending the target oligonucleotides by a thermostable DNA polymerase, and cleaving the extended products with a highly thermostable restriction endonuclease. PECR is able to amplify a specific oligonucleotide exponentially by only one probe instead of a pair of primers that is generally required in traditional nucleic acid amplification methods. The process of PECR is controlled by thermocycling. The parameters of the thermal cycles are flexibly adjustable according to the length, sequence, melting temperature and initial concentration of the target oligonucleotide. The reaction rate depends totally on the initial concentration of the target oligonucleotide in the reaction mixture. The method can amplify and quantify a specific small nucleotide acid, such as oligonucleotide or microRNA, from a small biology sample. PECR amplification of target oligonucleotides is conducted by thermocycling and without using a universal primer, it is thus simple, stable, efficient, and with high specificity, and thus can be widely useful in molecular biology studies.

The present invention is implemented by the following protocol: a method of polymerase-endonuclease chain reaction for the amplification of oligonucleotides and small RNAs, the method comprises:

1) The composition of the reaction mixture:

(1) A target nucleic acid sequence X, either double-stranded or single-stranded, length of 8 to 50 bases or base pairs, and its melting temperature (Tm) in the range of 36˜79° C.;

(2) An antisense probe, denoted by X′R′X′, is designed to be a single-stranded oligonucleotide containing at least two tandem repeated complements of the target sequence (X′) that are separated from one another by an intervening complementary recognition site (R′) for a restriction endonuclease;

(3) A thermostable DNA polymerase;

(4) A thermostable restriction endonuclease;

(5) Four deoxyribose nucleotides triphosphate: dATP, dGTP, dCTP and dTTP;

(6) An appropriate buffer solution;

2) The thermocycling reaction: the above reaction mixture is incubated at 60° C. to 99° C. for 0˜600 seconds of pre-denaturation, then subject to 1-100 cycles of thermocycling, each thermal cycle consists the following four steps:

(1) Denaturing: incubate the reaction mixture in a temperature at least 5° C. above the melting temperature of the target nucleic acid. The temperature ranges from 60˜99° C., duration ranges from 1 to 60 seconds;

(2) Annealing: incubate the reaction mixture in a temperature equal to, or within 5° C. higher or lower than, the melting temperature of the target nucleic acid. The temperature ranges from 35˜68° C., duration ranges from 1 to 60 seconds;

(3) Elongation: incubate the reaction mixture in a temperature at least 5° C. above the melting temperature of the target nucleic acid, and within the optimal working temperature of the said DNA polymerase. The temperature ranges from 45 to 89° C., duration ranges from 1 to 60 seconds;

(4) Cleaving: Insulation of the reaction mixture in a temperature at least 5° C. above the melting temperature of the target nucleic acid, and within the optimal operating temperature of the restriction enzymes the said. The temperature ranges from 45˜89° C., duration ranges from 1˜300 seconds;

The temperatures of (1) denaturing, (3) elongation and (4) cleaving steps are at least 10° C. higher than the annealing temperature in step (2). By repeated steps (1) to (4), say denaturation, annealing, extension and cleaving, the target nucleic acid molecules are amplified exponentially, the products include double-stranded repetitive nucleic acid XRX/X′R′X′, double-stranded target nucleic acid X/X′ and single-stranded target molecule X.

In this invention, the said thermostable DNA polymerase could resist high temperatures above 80° C. The best suited DNA polymerase is a hot start thermostable DNA polymerase. The said thermostable endonuclease is a double-stranded restriction enzyme which could resist high temperatures above 80° C.

The said target nucleic acid could be any kind of natural or synthetic DNA molecule, including oligonucleotides, genomic DNA, mitochondrial DNA, cDNA derived from reverse transcription of mRNA, microRNA, or siRNA, and so on. The said target nucleic acid could also be any type of synthetic or natural RNA molecules, including mRNA, microRNA and siRNA, and so on. If a DNA polymerase that can directly elongate RNA molecule, such as E. coli DNA polymerase I, is included in the reaction mixture, PECR reaction can also be used for direct amplification of RNA, particularly small RNAs, such as siRNA and miRNA.

The antisense probe may contain two or more tandem repeats of the complementary sequence (A′) of the target sequence (A). Between two adjacent repeats, there is at least one recognition sites (R′) of a thermostable endonuclease. The general molecular formula of the probe is A′-(R′A′)ₙ, where n is a positive integer greater than or equal to 1. Such kind of probe with multiple repeats enables faster rate per cycle of amplification.

The antisense probe may contain two or more different complementary target sequences (A′, B′, C′). Between two adjacent target sequences, there is at least one recognition sites (R′) of a thermostable endonuclease. The molecular formulas of the probe are A′-(R′B′)ₙ, B′R′A′-(R′ or A′R′B′—(R′C′)ₙ, where n is a positive integer greater than or equal to 1. Using such kind of probe containing different target sequences, different target sequences can be produced in a single PECR reaction. Moreover, one or more oligonucleotide inputs can be used to output another one or more target sequence(s), which can be useful in DNA circuit or DNA computing.

The end or the middle of the said antisense probe may contain one or more isotope labeled nucleotides, the labeled nucleotides can be introduced into the amplification product in random or predefined locations, so that the PECR product can be detected using radioactive detection methods.

The said reaction mixture may contain a DNA-specific fluorescent dye, including but not limited to Sybr Green I and Sybr Green II, so that the fluorescence intensity of the reaction mixture enhances with each round of PECR amplification, and the fluorescence signal can be detected by real-time fluorescence quantitative PCR instruments, and thus the initial amount of the target oligonucleotide can be quantitatively measured.

The middle or the end of the probe can be connected to one or more chemical groups, including but not limited to, fluorophores, quenching group, biotin, digoxin, amino acids, amino, amino-C3, amino-C6, amino-C12, amino-C18, Tsuen base, carboxyl, sugar ring, peptides, peptide nucleic acid, and so on.

The end or the middle of the said probe may contain a fluorophore and quencher groups, the fluorophore are located on one, and the quencher is on another, side of the restriction site. When the restriction sites were cleaved in a PECR reaction that makes the fluorophore and quencher separate from each other, the fluorescence intensity of the reaction mixture increased, which can be monitored by a real-time quantitative PCR instrument, so that the initial copy number of the target oligonucleotide can be quantitatively analyzed.

The end or the middle of the said target oligonucleotide may contain a fluorophore and a quencher group. When the restriction sites were cleaved in a PECR reaction that makes the fluorophore and quencher separate from each other, the fluorescence intensity of the reaction mixture increased, which can be monitored by a real-time quantitative PCR instrument, so that the initial copy number of the target oligonucleotide can be quantitatively analyzed.

The restriction sites in the said probe could be methylated, so that the restriction site could not be cut by endonuclease, but it could be cut after being demethylated. Note that in the PECR products the restriction sites could usually be cut because they are not methylated.

The said probe can be fixed in a gene chip, or the surface of solid materials, particles or plates, so that a large number of different target oligonucleotides could be detected by a high throughput method. The matrix could be made by various materials including silicon materials such as silicon or silicon dioxide film, silicon substrate, silicon nanowires, conductive metals such as gold, platinum, carbon materials, such as graphite, carbon nanotubes, and conductive resin, and so on. Some materials can also be used in the form of particles or beads, in which the probe is connected to the surface of these materials, so that PECR reactions could occur on the surface of them.

The end of the said probe could be connected with nano-materials, so nano-materials can be used to detect PECR reaction, or use PECR reactions to control nano-materials. Nano-materials are kinds of materials with zero-dimensional, one-dimensional, two-dimensional, or three-dimensional structures, which are composed of ultra-fine structures with small size effects (the size are smaller than 100 nm, ranges 0.1-100 nm). The shapes of nano-materials include nanowires, nanorods, nanotubes, nanobelts, nano-particles, nano-film, nano-crystals, nano non-crystalline, nano-fibers, nano-bulk, etc. Nano-materials include but not limited to, carbon nanotubes, nano-fullerenes (such as carbon sixty), nano-ceramics, nano-metal particles, zinc oxide particles, nano silica, nano-titanium dioxide and iron oxide nanoparticles. Nano-materials also include bio-nano-materials, which are biological macromolecules, such as polypeptide chains, polysaccharides, amino-polysaccharides and nucleic acids, and so on.

PECR product can be detected by the polyacrylamide gel electrophoresis (PAGE). Preparing a non-denaturing polyacrylamide gel with a concentration of 12%˜15%, and 5˜15 cm in length, running through a 250˜300V electrophoresis for 20˜40 min, the DNA bands can be visualized by one of the following methods:

(1) Staining the gel with ethidium bromide dye, then observe and photograph the DNA bands with UV gel imaging system;

(2) Staining the gel with Sybr Green I or Sybr Green II dye, and then observe and photograph the DNA bands with UV gel imaging system;

(3) Reveal DNA bands by silver staining;

(4) Mix radioisotope labeled single deoxyribonucleotide into the PECR reaction system, then operate electrophoresis followed by autoradiography imaging.

Real-time fluorescence quantitative detection can also be performed on PECR product with the following two methods:

(1) Adding fluorescent dye directly into the reaction mixture:

Adding Sybr Green I or II fluorescent dye into the reaction, Sybr Green binds specifically with the minor groove of DNA with high affinity for double-stranded DNA (dsDNA), while its binding capacity with single-stranded DNA (ssDNA) is very low. At the beginning of PECR reaction, the probe is single-stranded, thus binds with the Sybr Green weakly, and the fluorescence intensity is at a relatively low level. During PECR reaction cycles, single-stranded probe were converted into double-stranded products. The fluorescence intensity enhance greatly due to Sybr Green dyes bind with double-stranded products, which can be detected with a fluorescence quantitative real-time PCR instrument, such as ABI 7500.

(2) Labeling PECR probe with fluorophore and quencher groups:

Since Sybr Green dyes binds with dsDNA nonspecifically, quantification of nucleic acids based on them have the false-positive problem: if a false-positive or a nonspecific amplification occurred, it is not distinguishable from a true positive reaction. For the purpose of more accurate quantitative detection, one can label the PECR probe with fluorophore and quencher.

Fluorophores that can be used include but are not limited to: 6-carboxyfluorescein (FAM), Tetrachlorofluorescein (TET), hexachlorofluorescein (HEX), N,N,N,N′-tetramethyl-6-carboxyrhodamine (TAMRA), 6-carboxy-X-rhodamine(ROX), 2′7′-dimethoxy-4′5′-dichloro-6-carboxyfluorescein (JOE), indodicarbocyanine 3 (Cy3), indodicarbocyanine 5 (Cy5), fluorescein isothiocyanate (FITC), 3-(-carboxy-pentyl)-3′-ethyl-5,5′-dimethyloxacarbocyanine (CyA); Texas Red, 6-carboxyrhodamine (R6G) etc. Quenchers include but not limited to TARMA, Iowa Black (IWB), etc.

The said target nucleic acid of PECR reaction can be any DNA molecules, including oligonucleotides, genomic DNA, mitochondrial DNA, cDNA reverse transcript from mRNA, microRNA, or siRNA, and any other DNA molecules. PECR reaction can also be used for amplifying RNA directly, particularly siRNA, miRNA or other small RNA molecules. For different target nucleic acids, the technical protocols used are as followed:

(1) For single-stranded or double-stranded oligonucleotides which has a length of 8 to 50 base pairs, use the basic PECR protocol to do amplification;

(2) For microRNA or siRNA, use the reverse transcript PECR (RT-PECR) protocol, which first reverse transcript the microRNA or siRNA into cDNA, and then conduct the amplification by PEAR, or adopt the RNA direct PECR (RD-PECR) protocol to amplify the target RNA directly;

(3) For a long nucleic acid which has more than 50 base pairs, this method can only amplify the specific sequence with the length about 8 to 50 base pairs in the 3′-end, but not the full sequence;

(4) For a specific sequence in the middle of a long target, firstly use an endonuclease, whose recognition site is closely adjacent to the target sequence, to cut the target DNA, letting the target sequence exposed to the 3′-end, and then amplify by PECR.

The present invention is the first to disclose the polymerase-endonuclease chain reaction (PECR), or polymerase-endonuclease amplification reaction (PEAR), which is a new nucleic acid amplification technology. The difference between PEAR and other nucleic acid amplification technology are as followed:

(1) The comparison of PECR and other existing DNA Amplification technologies: PCR amplify linear or circular DNA to single copy linear fragments through thermal cycling; RCA amplify circular DNA to linear multi-copy tandem repeat DNA molecules through isothermal reaction; LAMP amplify linear DNA to linear multi-copy tandem repeat DNA through isothermal reaction; EXPAR use tandem repeat DNA template to amplify oligonucleotides through isothermal reaction; PECR use tandem repeat DNA probe to amplify small nucleic acids through a thermal cyclic reaction, so the PECR technology presented in this invention is an important new member of the family of nucleic acid amplification technologies.

(2) The comparison between PECR and PCR technique: the principle of PECR is different from that of PCR, The major differences include: (1) PCR depends only on thermostable DNA polymerase, while PECR depends not only on thermostable DNA polymerase, but also on thermostable endonuclease; (2) PCR requires at least a pair of primers, but PECR needs only one single repeat-containing probe; (3) In a PCR reaction, primers are only extended, while in a PECR the repeat-containing products is not only extended but cleaved, so that the copy number of product molecules increases in each cycle; (4) PCR is not able to directly amplify nucleic acid whose length is too short, while PECR is designed to directly amplify nucleic acid of a shorter length, particularly oligonucleotides and small RNAs; (5) PCR products are usually longer than primers used, while PECR products are shorter than the probe; (6) In each cycle of PCR amplification, products can only be doubled at most, and the total number of final product molecules could not exceed the number of input primers, while in PECR using probes with more tandem repeats, products can be increased more than two-fold for every cycle, and the copy number of final product molecules far exceeds the copy number of input probes;

(3) The comparison between PECR and coupled PCR-restriction endonuclease digestion (PCR-RED): in patent PCT/US2000/007133 titled “COUPLED POLYMERASE CHAIN REACTION-RESTRICTION ENDONUCLEASE DIGESTION-LIGASE DETECTION REACTION PROCESS”, which described PCR-restriction endonuclease digestion, which can eliminate or significantly reduce the formation of non-specific PCR products. Although the reaction both adopt the DNA polymerase and thermostable restriction enzymes, the principles of PECR and PCR-RED are fundamentally different: the basic principle of the PCR-RED is still same to PCR and the thermostable restriction enzymes play only an assistant role. The purpose is to eliminate or reduce the non-target DNA amplification which contains restriction sites of thermostable restriction enzymes by enzyme digestion, but not to achieve exponentially amplification of the target DNA. While in PECR, the role of thermostable restriction enzyme is not to eliminate non-target DNA amplification, but the key enzymes to achieve exponentially amplification of the target DNA.

(4) The comparison between PECR methods and EXPAR Methods: In the patent “Isothermal reactions for the Amplification of oligonucleotides” (PCT/US04/02718) described an isothermal exponential amplification reaction that is called EXPAR reaction. Both the present invention PECR and EXPAR reaction use the same probe design strategy, but PECR reaction and EXPAR reaction are fundamentally different: (1) EXPAR is an isothermal amplification reaction, the process of EXPAR reaction is not controlled, while PECR reaction process is tightly controlled by thermal cycling; (2) EXPAR depends on a single strand nicking enzyme, while PECR adopts a double-stranded endonuclease; (3) EXPAR is not able to use a hot-start DNA polymerase, so that only manually hot start can be applied, while PECR can be automatically hot started by a thermalcycler using hot-start DNA polymerase; (4) EXPAR reaction has seriously non-specific background amplification and false-positive problem, while PECR reaction has little non-specific background amplification and can in principle overcome the false positive problem thanks to the tightly controlled thermal cycling.

Although Tan et al. reported a EXPAR reaction which was carried out by manually hot start to reduce non-specific amplification, but hot-starting of the EXPAR, do not like PCR, could not be implemented automatically by a PCR thermalcycler using hot-start DNA polymerase (Tan E, et al, Specific versus nonspecific isothermal DNA amplification through thermophilic polymerase and nicking enzyme activities. Biochemistry. 2008, 47 (38): 9987-9999). A hot start polymerase, which is a reversible inactivation of a DNA polymerase prepared through chemical modification or anti-polymerase antibody, could not be used in EXPAR. Because the hot start polymerase must be heat activated at above 90° C. for about 10 min, while EXPAR reaction depends on a strand displacement activity of the Bst polymerase and the nicking enzyme Nb. BstNBI, which would be both heat inactivated in such a high temperature. Although there are some strand displacement DNA polymerases (such as VentR exo-) that could resist high temperature above 90° C., but at present highly thermostable nicking enzyme, which can resist high temperatures above 90° C., is not available. Therefore, the EXPAR reaction must be hot start manually: first heat reaction mixture to a predetermined temperature, then add the DNA polymerase and the nicking enzyme. Manual hot start is not only cumbersome, but could not be implemented for real time quantitative analysis, greatly limited the application of the method.

In contrast, PECR reactions can be conducted using a hot start DNA polymerase, so that automatic hot started by a PCR instrument with the inherent reliability and convenience. In addition, as in the PCR reaction, the PECR process is tightly controlled by thermal cycling, and the reaction parameters including annealing temperature, annealing time, number of cycles, and so on, is flexibly adjustable according to the length, sequence, melting temperature, and the initial number of the target oligonucleotides.

In summary, PECR is a new, simple but effective nucleic acid amplification technology. By PECR, using a specific probe, we can selectively amplify a specific small nucleic acid, including oligonucleotides and miRNAs, with a known sequence quantitatively, rapidly, accurately and sensitively. PECR is easy to adapt for fully automation and real-time quantitative detection, thus could be widely useful in molecular biology studies, e.g. amplification and quantification of miRNAs and small RNAs for gene expression profiling, gene chip technologies, high-throughput nucleic acid detection, large-scale amplification or preparation of antisense oligonucleotides, intelligent nucleic acid detection and molecular computing, etc.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1. The schematic diagram of polymerase-endonuclease chain reaction;

FIG. 2. The schematic diagram of fluorescent labeling of PECR probe;

FIG. 3. Verification of PECR reaction principle (Embodiment 1);

FIG. 4. Electrophoresis result of amplification with different initial concentration of target oligonucleotides (Embodiment 1);

FIG. 5. The schematic diagram of reverse transcriptase PECR (RT-PECR);

FIG. 6. The schematic diagram of RNA-direct PECR (RD-PECR);

FIG. 7. A result of real-time fluorescence detection of PECR products (Embodiment 6).

### DETAILED DESCRIPTION OF THE PREFERRED EMBODIMENT

### Examples of Embodiment

### Embodiment 1

In this example of embodiment, polymerase-endonuclease chain reaction is implemented to amplify oligonucleotides using thermostable DNA polymerase and thermostable restriction endonuclease that can cut double-stranded DNA. The said thermostable DNA polymerase and thermostable endonuclease can resist high temperature above 50° C., and its optimum working temperature range is 45-89° C. The thermostable DNA polymerase includes but not limits to Taq DNA polymerase, DyNAzyme II DNA Polymerase®, LA Taq DNA Polymerase®, Pfu DNA polymerase ®, VentR DNA Polymerase®, Deep VentR DNA Polymerase®, VentR exo-DNA Polymerase®, Deep VentR (exo-) DNA Polymerase®, 9° Nm DNA Polymerase®, etc. A hot-start DNA polymerase will be better for use in this reaction. Hot-start DNA polymerases include but not limits to hot-start Taq DNA polymerase, DyNAzyme II Hot Start DNA Polymerase®, KOD Xtreme Hot Start DNA Polymerase®, Phusion DNA Polymerase®, Pfu Ultra type of hot start DNA Polymerase®, Platinum DNA polymerase and Thermo-Start DNA Polymerase®, etc. Thermostable restriction enzymes include but not limited to PspGI, ApeKI, BstUI, BstNI, MwoI, Phol, TseI, Tsp451, Tsp5091, TspRI and TfiI, etc.

The method comprises the following:

1) The composition of the reaction mixture:

(1) A target nucleic acid X, either double-stranded or single-stranded, length is in the range of 8 to 50 bases or bp, and its melting temperature is in the range of 36˜79° C.;

(2) An antisense probe, denoted by X′R′X′, is designed to be a single-stranded oligonucleotide containing at least two tandem repeats of complement target sequence (X′) that are separated from one another by an intervening complementary recognition site (R′) for a restriction endonuclease;

(3) A thermostable DNA polymerase;

(4) A thermostable restriction endonuclease;

(5) Four deoxyribose nucleotides triphosphate: dATP, dGTP, dCTP and dTTP;

(6) An appropriate buffer solution;

2) The thermocycling reaction: the above reaction mixture is incubated at 60° C. to 99° C. for 0˜600 seconds of pre-denaturation, then subject to 1-100 cycles of thermocycling, each thermal cycle consists the following four steps:

(1) Denaturing: incubate the reaction mixture in a temperature at least 5° C. above the melting temperature of the target nucleic acid. The temperature ranges from 60˜99° C., duration ranges from 1 to 60 seconds;

(2) Annealing: incubate the reaction mixture in a temperature equal to, or within 5° C. higher or lower than, the melting temperature of the target nucleic acid. The temperature ranges from 35˜68° C., duration ranges from 1 to 60 seconds;

(3) Elongation: incubate the reaction mixture in a temperature at least 5° C. above the melting temperature of the target nucleic acid, and within the optimal working temperature of the said DNA polymerase. The temperature ranges from 45 to 89° C., duration ranges from 1 to 60 seconds;

(4) Cleaving: Insulation of the reaction mixture in a temperature at least 5° C. above the melting temperature of the target nucleic acid, and within the optimal operating temperature of the restriction enzymes the said. The temperature ranges from 45˜89° C., duration ranges from 1˜300 seconds;

The temperatures of (1) denaturing, (3) elongation and (4) cleaving steps are at least 10° C. higher than the annealing temperature in step (2). By repeated steps (1) to (4), say denaturation, annealing, extension and cleaving, the target nucleic acid molecules are amplified exponentially, the products include double-stranded repetitive nucleic acid XRX/X′R′X′, double-stranded target nucleic acid X/X′ and single-stranded target molecule X.

In practice, if the temperature of cleaving is the same with that of extension, then step (4) and (3) can be combined into one single step: Step (3) extension and cleaving, and the duration ranges between 1-300 sec.

The schematic diagram of the mechanism of PECR amplification reactions is shown in FIG. 1: Sense and antisense strands are represented by solid and dashed lines respectively, the 3′-ends are indicated by arrows and the restriction sites for PspGI are indicated by solid diamonds. When a target oligonucleotide (X) binds to a probe in the upstream, it is elongated by the Taq DNA polymerase, and a full-duplex oligonucleotide containing tandem repeats is produced. If the repeats are cleaved by PspGI, short duplex target oligonucleotides (MC) are released; and when they are not cleaved, the number of tandem repeats increases by slipping and elongation. As shown in FIG. 1, PEAR consists of repetitive cycles of: (1) heat denaturation, (2) annealing, (3) elongation, and (4) cleaving. In the first cycle, a target oligonucleotide and an antisense probe were heat-denatured and annealed to form a partial duplex (X/X′R′X′). In the presence of dNTPs, they are elongated by Taq DNA polymerase to form fully matched duplex tandem repeats (XRX/X′R′X′). Subsequently, PspGI cleavage of the recognition site releases monomeric oligonucleotides (X/X′). Thereafter, a next cycle of denaturation, annealing, elongation and cleaving is started again, resulting in exponential amplification of the duplex oligonucleotide, and the amplification product is the double-stranded target molecule X/X′.

In the step (2), when a target oligonucleotide binds to a probe in the upstream (FIG. 1, top right), there is no elongation, because it provides no primer/template structure for the Taq DNA polymerase. This would affect the kinetics of PECR, but would not lead to the stop of the PECR amplification because of the following two reasons: (1) Both target and probe are present in a large number of copies, according to the law of probability, nearly half of the target oligonucleotides bind to the probe in the downstream, and start the reaction; (2) Even if only one target molecule is presented in the reaction, with several cycles of denaturation and annealing, it will eventually bind to the probe's in the 3′-end, and start the reaction.

In addition, in step (3) and (4), in the subsequent thermal cycles, if the tandem repeated duplexes are not fully digested by PspGI, because the duration of cleavage is rather short. When the remaining tandem repeated duplexes are subjected to more cycles of denaturing, reannealing and elongation, the number of repeat unit increases continuously through slipped strand pairing and DNA polymerase elongation (FIG. 1, bottom right). The slipping reaction is linear, which can have an impact on the kinetics and the rate of amplification of PECR reaction. However, provided sufficient amount of restriction enzymes, most of the duplex repeats will be cut, so it will not affect the exponential feature of the PECR reaction.

In fact, when PspGI cleavage monomerizes the elongated tandem repeats in a following cycle, many more duplex oligonucleotides are released. It is this slipping-and-cleaving mechanism that promotes not only the rate of amplification, but also the yield of product. To facilitate the analysis and detection, or applying in a subsequent reaction, if necessary, the PEAR product is finally cleaved by PspGI for 10˜60 minutes after the completion of the thermal cycles, monomerizes the tandem repeats fully into duplex oligonucleotides.

PECR product can be detected by the polyacrylamide gel electrophoresis (PAGE). Preparing a non-denaturing polyacrylamide gel with a concentration of 12%˜15%, and 5˜15 cm in length, running through a 250˜300V electrophoresis for 20˜40 min, the DNA bands can be visualized by one of the following methods:

(1) Staining the gel with ethidium bromide dye, then observe and photograph the DNA bands with UV gel imaging system;

(2) Staining the gel with Sybr Green I or Sybr Green II dye, and then observe and photograph the DNA bands with UV gel imaging system;

(3) Reveal DNA bands by silver staining;

(4) Mix radioisotope labeled single deoxyribonucleotide into the PECR reaction system, then operate electrophoresis followed by autoradiography imaging.

Real-time fluorescence quantitative detection can also be performed on PECR product with the following two methods:

(1) Adding fluorescent dye directly into the reaction mixture:

Adding Sybr Green I or II fluorescent dye into the reaction, Sybr Green binds specifically with the minor groove of DNA with high affinity for double-stranded DNA (dsDNA), while its binding capacity with single-stranded DNA (ssDNA) is very low. At the beginning of PECR reaction, the probe is single-stranded, thus binds with the Sybr Green weakly, and the fluorescence intensity is at a relatively low level. During PECR reaction cycles, single-stranded probe were converted into double-stranded products. The fluorescence intensity enhance greatly due to Sybr Green dyes bind with double-stranded products, which can be detected with a fluorescence quantitative real-time PCR instrument, such as ABI 7500.

(2) Labeling PECR probe with fluorophore and quencher groups:

Since Sybr Green dyes binds with dsDNA nonspecifically, quantification of nucleic acids based on them have the false-positive problem: if a false-positive or a nonspecific amplification occurred, it is not distinguishable from a true positive reaction. For the purpose of more accurate quantitative detection, one can label the PECR probe with fluorophore and quencher.

Fluorophores that can be used include but are not limited to: 6-carboxyfluorescein (FAM), Tetrachlorofluorescein (TET), hexachlorofluorescein (HEX), N,N,N;N′-tetramethyl-6-carboxyrhodamine (TAMRA), 6-carboxy-X-rhodamine(ROX), 2′7′-dimethoxy-4′5′-dichloro-6-carboxyfluorescein (JOE), indodicarbocyanine 3 (Cy3), indodicarbocyanine 5 (Cy5), fluorescein isothiocyanate (FITC), 3-(-carboxy-pentyl)-3′-ethyl-5,5′-dimethyloxacarbocyanine (CyA); Texas Red, 6-carboxyrhodamine (R6G) etc. Quenchers include but not limited to TARMA, Iowa Black (IWB), etc.

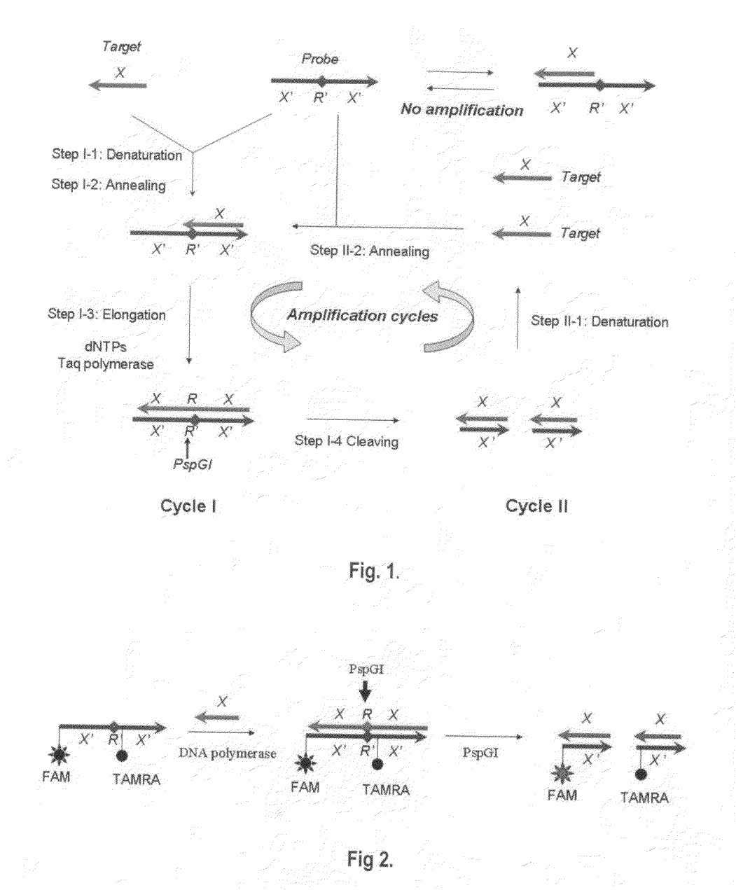

The principle of fluorescent labeling is shown in FIG. 2, in which the fluorophore lies in the 5′-end of the probe, while the quencher locates in the middle of which, more precisely, 3-10 bases downstream the restriction enzyme cleavage site R′. At the beginning of the PECR reaction, the fluorescence is close to the quencher, according to the fluorescence resonance energy transfer (FRET) principle most of the energy absorbed by the fluorophore transfers to the quencher and releases as heat, therefore fluorescence occurred in a lower level. Note that if the quencher was set on the 3′-end of the probe, they would be too far apart from each other, which may cause the quenching to be ineffective. As PECR reaction occurs, single-stranded probes are continually being converted into double-stranded products and being cut by restriction enzymes, leading to the separation of the fluorophore and the quencher. Energy absorbed by fluorophore will be released in the form of fluorescence, with its signal being greatly enhanced, which can therefore be detected by the fluorescence quantitative real-time PCR instrument.

Specifically, all the dNTPs, DyNAzyme II Hot Start DNA polymerase, restriction enzymes PspGI and buffer solution used in this procedure are purchased from New England Biolabs, Co. Ltd, Beijing branch. The synthetic oligonucleotides and probes are purchased from Invitrogen Co. Ltd, Shanghai branch. The target oligonucleotides (X) is derived from a human microRNA, hsa-miR-375, its sequence is: 5′-TTTGTTCGTTCGGCTCGCGTGA-3′. In order to fasten the rate of amplification, the probe (X′R′X′R′X′) we adopted contains 3 copies of the complementary sequence of hsa-miR-375, which is:

5′-TCACGCGAGCCGAACGAACAAA-CCAGG-TCACGCGAGCCGAACGA
ACAAA-CCAGG-TCACGCGAGCCGAACGAACAAA-3′

The underlined shows the enzyme PspGI recognition and cleaving sites.

In a 20 μL volume reaction mixture, add 100-200 nM of probe (X′R′X′R′X′), 10⁻¹ to 10⁻¹² uM of target oligonucleotides (X), 0.02-0.1 Unit/uL of DyNAzyme II Hot Start DNA Polymerase, 0.01-0.5 Unit/μL of restriction enzyme PspGI, 1× of DyNAzyme II Hot Start DNA polymerase buffer and 50 uM each dNTPs. The reactions were initiated at 90-95° C. for 1-10 min for hot start and activation of the DNA polymerase DyNAzyme II, followed by 20-40 cycles of denaturing at 90-95° C. for 5-30 sec, annealing at 45-65° C. for 5-30 sec, elongation and cleaving at 75° C. for 1-5 min. If necessary, PspGI digestion of the product is conducted by a final incubation at 75° C. for 10-60 min. PEAR products were separated by 15% non-denaturing polyacrylamide gel electrophoresis (PAGE), and visualized under an ultraviolet illuminator after SYBR Gold staining, which is purchased from Molecular Probes.

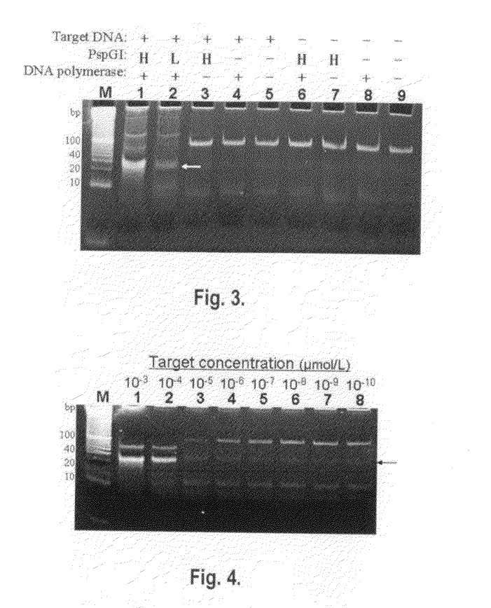

To validate the reaction mechanism, PEAR reactions with complete and incomplete (lacking Taq DNA polymerase, PspGI or target) components were conducted under previously optimized reaction conditions with target concentration at 1 nM and probe concentration at 100 nM. As indicated by the arrow in FIG. 3, a lower band (22 bp), representing the duplex monomers, X/X′, and several upper bands, representing tandem repeats, are observed in the complete PEAR reactions. In addition, a higher concentration of restriction enzyme (H in FIG. 3) produces a stronger band than a low concentration of enzyme (L in FIG. 3). Such bands are not observed if any of the four essential components (the two enzymes, the target and the probe) is omitted. It is clear that the PECR amplification depends on both of the two enzymes, the probe and the target nucleic acid. FIG. 4 shows that PECR amplification is highly sensitive and can detect target oligonucleotide as low as 10⁻¹⁰ μM.

### Embodiment 2

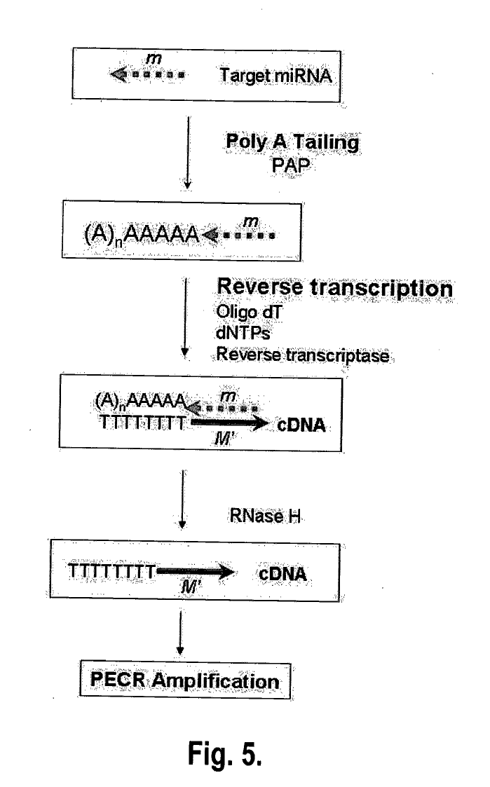

This embodiment is an example of reverse transcriptase PECR (RT-PECR). RNA molecules, particularly small RNAs such as miRNA or siRNA, were amplified by RT-PECR. Take miRNA as an example, the reaction principle is shown in FIG. 5. The method comprises the following steps:

Add poly-A tail to total miRNA using poly-A polymerase (PAP);

Reverse transcript total miRNA to cDNA using Oligo-dT and reverse transcriptase;

Remove RNA molecules from the product cDNA using RNase H;

Amplify the target cDNA using PECR, the components of the reaction mixtures and the thermal cycling parameters are the same as those in embodiment 1.

### Embodiment 3

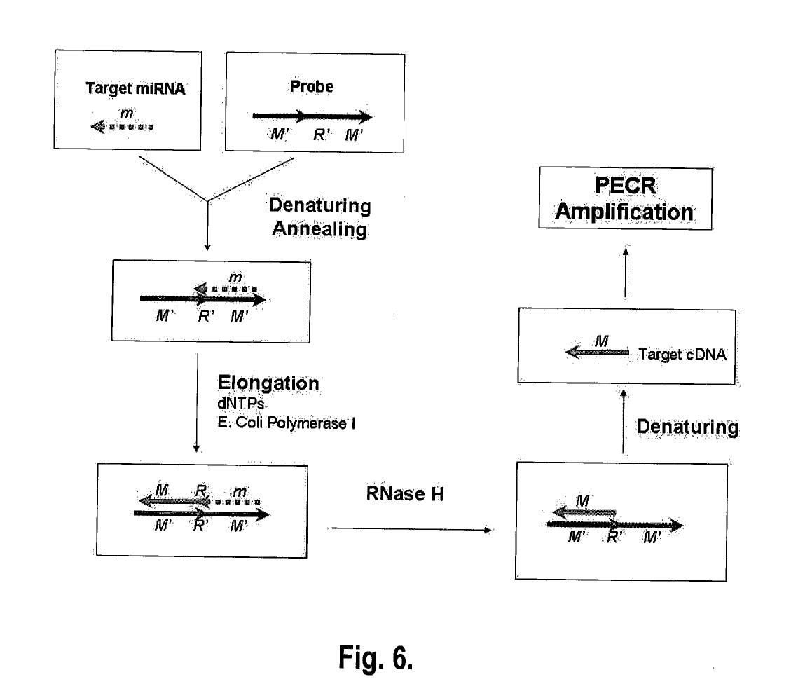

This embodiment is RNA-direct PECR (RD-PECR), i.e., directly amplify RNA molecules by PECR without reverse transcription. Take miRNA as an example, the reaction principle is shown in FIG. 6, and the method comprises the following four steps:

(1) Mix the PECR probe with total RNA directly, by heat denaturation and annealing, the target miRNA bind to PECR probe to form partial duplex miRNA/DNA hybrid molecules;

(2) Adding in the reaction mixture a DNA polymerase which can directly extend RNA molecules, e.g., E. coli DNA polymerase I, setting the temperature of the first thermal cycle to be the optimum working temperature for the DNA polymerase I (37° C.), miRNA strands in the partially duplexes are extended at their 3′-end, forming target cDNA molecules whose sequences are the same to the target miRNA;

(3) Remove RNA molecules from the product cDNA using RNase H;

(4) Amplify the target cDNA using PECR, the components of the reaction mixtures and the thermal cycling parameters are the same as those in embodiment 1.

### Embodiment 4

Choose 4 zebrafish miRNA as target, respectively miR-375, miR-430a, miR-206 and miR-124. The sequence and function of miR-375 and miR-430a are known, they are selected for technical verification. MiR-375 is necessary for pancreatic development, reducing the level of miR-375 can inhibit the aggregation of islet cells. The function of miR-430 is to clear maternal mRNAs that are no longer needed in zebrafish embryos. In addition, it has been reported that peaks of miR-430a, miR-206 and miR-124 expression appeared respectively at 4 h, 12 h and 24 h after fertilization in zebrafish embryos. This Embodiment performs a comparison of RT-PCR and RT-PECR through analysis of miRNA expression in zebrafish early embryo development.

(1) Total RNA Extraction and Reverse Transcription

Using Applied Biosystems mirVana miRNA Isolation Kit (Cat #AM1560), total miRNA of zebrafish early embryos at 1 h, 2 h, 4 h, 12 h and 24 h after fertilized was extracted. Using Applied Biosystems TaqMan miRNA Reverse Transcription Kit (Cat # 4366596), the total miRNA was reverse transcript into cDNA, and used as template in subsequent PCR and PECR reactions.

(2) Real-Time Quantitative PCR

Using glycerol 3-phosphate dehydrogenase (GAPDH) gene as internal control, using Applied Biosystems TaqMan MicroRNA Assay (Cat #4383443) and TaqMan Universal PCR Master Mix (Cat #4364338) for quantitative detection of the target miRNA in the reverse transcription products, the samples were standardized, and used as external controls for PECR reactions.

(3) Real-Time Quantitative PECR

Using real-time quantitative PECR for quantitative detection of the target miRNA in the samples, the components of the reaction mixture and thermal cycling parameters are the same as those in embodiment 1, except that the probes are labeled with a fluorophore and a quencher. All reactions include a no-template control (NTC), and were repeated three times at least. The reactions were conducted in the Applied to Biosystems 7500 Real-Time PCR system, and the fluorescence intensities were monitored in real-time as PECR cycle changes.

(4) Results and Analysis

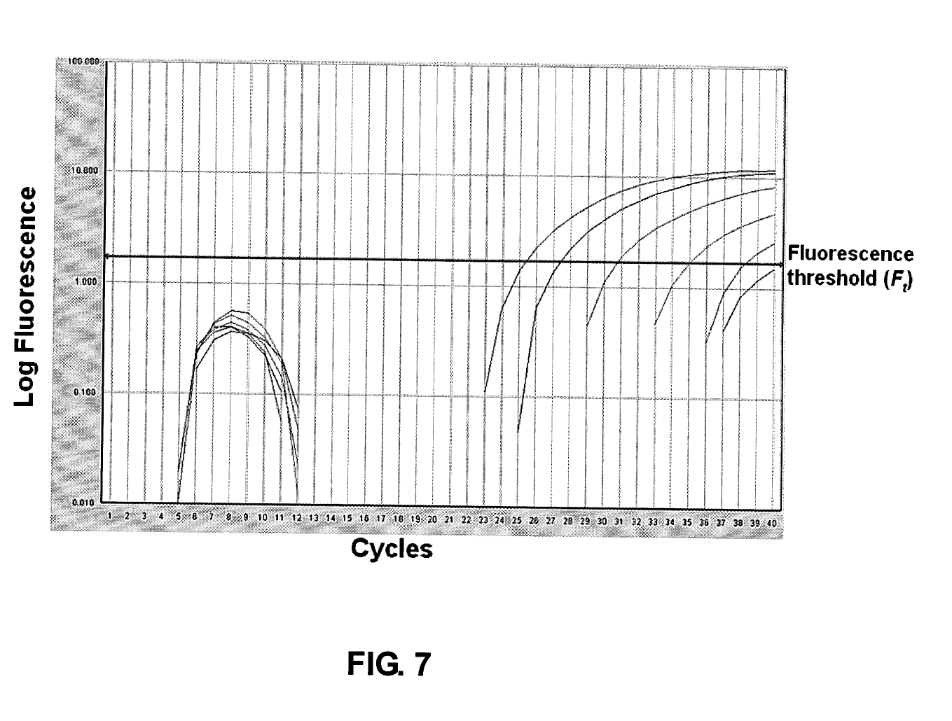

As shown in FIG. 7, real-time fluorescence detection confirmed that fluorescence intensities increased with each PECR cycle. Comparing the results of RT-PCR and RT-PECR, it was shown that they have basically consistent results, indicated the high accuracy of real-time quantitative PECR.

A universal primer must be used to match a miRNA-specific primer when amplify miRNA by RT-PCR. It will possibly cause non-specific amplification, false positives and false negatives issues. However, when the detection is done by RT-PECR, only a repeat-containing probe that is complementary to the target miRNA is needed. Therefore, the PECR technology has characteristics of simple, efficient and stable, and with higher specificity. So PECR is potentially useful in amplification and quantitative analysis of miRNA.

Throughout in this description and in the claims, the singular may include the plural unless clearly stated. For example, adding “a thermostable enzyme” in the reaction system includes adding one or more kinds of thermostable enzymes; adding “a thermostable DNA polymerase” in the reaction system concludes adding one or more kinds of thermostable DNA polymerases; “a target molecule” includes one or more target molecules; “a probe” includes one or more probes, and so on.

In addition, this invention is not limited to the particular configuration of the description. The terminology in the description and the claims are only used for the description of a specific implementation, but not to limit the invention to the qualified range of terminology used, because the scope of the invention is restricted only by the claims of rights and requirements or articles which are equal to them.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A polymerase-endonuclease chain reaction for the amplification of oligonucleotides and small RNAs. The method comprising:
1) The composition of the reaction mixture:
(1) A target nucleic acid sequence X, either double-stranded or single-stranded, length of 8 to 50 bases or base pairs, and its melting temperature (Tm) in the range of 36˜79° C.;
(2) An antisense probe, denoted by X′R′X′, is designed to be a single-stranded oligonucleotide containing at least two tandem repeated complements of the target sequence (X′) that are separated from one another by an intervening complementary recognition site (R′) for a restriction endonuclease;
(3) A thermostable DNA polymerase;
(4) A thermostable restriction endonuclease;
(5) Four deoxyribose nucleotides triphosphate: dATP, dGTP, dCTP and dTTP;
(6) An appropriate buffer solution;
2) The thermocycling reaction: the above reaction mixture is incubated at 60□ to 99□ for 0˜600 seconds of pre-denaturation, then subject to 1-100 cycles of thermocycling, each thermal cycle consists the following four steps:
(1) Denaturing: incubate the reaction mixture in a temperature at least 5□ above the melting temperature of the target nucleic acid. The temperature ranges from 60˜990, duration ranges from 1 to 60 seconds;
(2) Annealing: incubate the reaction mixture in a temperature equal to, or within 5□ higher or lower than, the melting temperature of the target nucleic acid. The temperature ranges from 35˜68□, duration ranges from 1 to 60 seconds;
(3) Elongation: incubate the reaction mixture in a temperature at least 5□ above the melting temperature of the target nucleic acid, and within the optimal working temperature of the said DNA polymerase. The temperature ranges from 45 to 89□, duration ranges from 1 to 60 seconds;
(4) Cleaving: Insulation of the reaction mixture in a temperature at least 5□ above the melting temperature of the target nucleic acid, and within the optimal operating temperature of the restriction enzymes the said. The temperature ranges from 45˜89□, duration ranges from 1˜300 seconds;
the temperatures of (1) denaturing, (3) elongation and (4) cleaving steps are at least 10□ higher than the annealing temperature in step (2). By repeated steps (1) to (4), say denaturation, annealing, extension and cleaving, the target nucleic acid molecules are amplified exponentially, the products include double-stranded repetitive nucleic acid XRX/X′R′X′, double-stranded target nucleic acid X/X′ and single-stranded target molecule X.

2. The method of claim 1 wherein the said DNA polymerase is a hot start thermostable DNA polymerase. The said restriction enzyme is a thermostable double-stranded endonuclease.

3. The method of claim 1 wherein the said target nucleic acid is any synthetic or natural DNA molecules, including oligonucleotides, genomic DNA, mitochondrial DNA, cDNA derived from reverse transcription of mRNA, microRNA, or siRNA.

4. The method of claim 1 wherein the said target nucleic acid is RNA molecules, including mRNA, microRNA and siRNA, or any other kind of synthetic or natural RNA molecules, and a DNA polymerase that can elongate RNA molecules directly is included in the said reaction mixture.

5. The method of claim 1 wherein the said antisense probe contains two or more tandem repeats of the complementary sequence (A′) of the target sequence (A). Between two adjacent repeats, there is at least one recognition sites (R′) of a thermostable endonuclease. The general molecular formula of the probe is A′-(R′A′)ₙ, where n is a positive integer greater than or equal to 1.

6. The method of claim 1 wherein the said antisense probe contains two or more different complementary target sequences (A′, B′, C′). Between two adjacent target sequences, there is at least one recognition sites (R′) of a thermostable endonuclease. The molecular formulas of the probe are A′-(R′B′)ₙ, B′R′A′—(R′ B′)ₙ, or A′R′B′—(R′C′)ₙ, where n is a positive integer greater than or equal to 1.

7. The method of claim 1 wherein the end or the middle of the said probe contains an isotope labeled nucleotides.

8. The method of claim 1 wherein the said the reaction mixture contains a DNA-specific binding fluorescent dye.

9. The method of claim 1 wherein the end or the middle of the said probe in connection with one or more chemical groups.

10. The method of claim 9 characterized in that one of the said chemical groups is fluorophores.

11. The method of claim 1 wherein the end or the middle of the of the said target nucleic acid containing fluorophores.

12. The method of claim 1 wherein the said probe is methylated.

13. The method of claim 1 wherein the said probe is fixed on a gene chip or other solid surface.

14. The method of claim 1 wherein the end of the said probe is connected to a nano-material.
